(19) United States
(12) Patent Application Publication
(10) Pub. No.: US 2011/0070177 A1
(43) Pub. Date: Mar. 24, 2011

(54) COSMETIC PACKAGING AND APPLICATION COMBINATION FOR SHEAR-THINNING COMPOSITIONS

(75) Inventors: Pascal ARNAUD, L'Hay Les Roses (FR); Marie Vidal, Castres (FR)

(73) Assignee: L'OREAL, Paris (FR)

(21) Appl. No.: 12/885,656

(22) Filed: Sep. 20, 2010

Related U.S. Application Data
(60) Provisional application No. 61/263,874, filed on Nov. 24, 2009.

(30) Foreign Application Priority Data
Sep. 21, 2009 (FR) 09 56483

(51) Int. Cl. B65D 69/00; A61K 8/18; A61Q 1/02

(52) U.S. Cl. 424/63; 206/581

(57) ABSTRACT

The present invention relates to a cosmetic packaging and application combination for making up and/or caring for keratinous substances including 1) a container holding a cosmetic composition in the water-in-oil emulsion form and characterized by a certain rheological profile and 2) an applicator.

### REFERENCE TO PRIOR APPLICATIONS

This application claims priority to U.S. provisional application Ser. No. 61/263,874, filed Nov. 24, 2009; and to French patent application 09 56483, filed Sep. 21, 2009, both incorporated herein by reference.

### FIELD OF THE INVENTION

The present invention is targeted at providing a novel method for making up and/or caring for keratinous substances more particularly dedicated to the application, to a keratinous substance, in particular to the skin, of fluid cosmetic compositions in the form of water-in-oil emulsions.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. In this regard, the description herein is to be understood as illustrative in nature, and not as restrictive.

The term “keratinous substances” according to the invention is understood to mean in particular the skin and the lips, especially the skin, and in particular the skin of the face. Other keratinous substances are also included.

### BACKGROUND OF THE INVENTION

Foundation compositions are commonly employed to give the skin, in particular the face, an attractive colour but also to conceal imperfections of the skin, such as red blotches and/or blemishes. More particularly, in the field of makeup and/or care compositions with a fluid texture, it is commonplace to use compositions in the form of water-in-oil (W/O) emulsions comprising an aqueous phase dispersed in an oily phase.

Consumers are increasingly looking for products which are easy and fast to apply, which do not make the hands messy and which produce a layer deposited on the skin which is as homogeneous as possible.

The practicality of the application is all the more desired when the product is liquid as, in this case, it is more problematic to grasp it than to apply it to the skin. Specifically, the product can then be difficult to measure out, can fall between the fingers and can stain the clothing.

In the case of a makeup product, such as liquid foundations, consumers are obliged to wash their hands after application, which is a loss in time and sometimes cannot be carried out under good conditions, in particular in conveyances.

Homogeneity is also an important criterion which is not always met when the product is applied with the fingers. In point of fact, this homogeneity of the deposited layer is fundamental, both aesthetically, for a makeup product, and with regard to effectiveness, for an antisun product or a care product.

In order to facilitate application, is it known to a person skilled in the art to use applicators, such as brushes or sprays. However, the latter do not make it possible to obtain an optimum homogeneity of the deposited layer.

The hairs of the brush can leave streaks during application and sprays result in the deposited layer having a thickness which can vary greatly from one spot to another on the face or on the body depending on the body movements during application.

### SUMMARY OF THE INVENTION

It thus remains necessary to look for a technical solution which makes it possible to obtain both good practicality and a homogeneous deposited layer on application.

The present invention is targeted precisely at meeting this need.

### BRIEF DESCRIPTION OF THE DRAWINGS

FIG. 1 diagrammatically represents, in perspective, an example of a packaging and application combination in accordance with the invention,

FIG. 2 represents, in partial and diagrammatic longitudinal section in the Y axis, the example of a packaging and application combination of FIG. 1 in accordance with the invention,

FIG. 3 represents, in partial and diagrammatic longitudinal section in the Y axis, an alternative form of the packaging and application combination in accordance with the invention (with piston).

### DETAILED DESCRIPTION OF THE PREFERRED EMBODIMENTS

In the following description certain features or elements of the accompanying drawings are referred to in order to further illustrate the invention. In all instances these references simply show example embodiments of, and do not limit, the identified element or feature.

In more detail, the present invention relates, according to one of its aspects, to a cosmetic packaging and application combination for making up and/or caring for keratinous substances comprising at least:

one container comprising at least one cosmetic composition in the water-in-oil emulsion form, characterized by the following rheological profile:
a viscosity of between 35 and 1000 Pa·s, in particular between 40 and 950 Pa·s, preferably between 55 and 900 Pa·s, at a shear gradient of 0.01 s⁻¹,
a viscosity of between 0.20 and 0.90 Pa·s, in particular between 0.25 and 0.80 Pa·s, preferably between 0.28 and 0.70 Pa·s, at a shear gradient of 1000 s⁻¹,
   as measured using a controlled-stress rheometer, AR-G2, TA Instruments, at 25° C., and

an applicator head (3) integral with the said container and supporting an application means (7), the said application means comprising an application surface capable, in response to its movement on engaging with the keratinous substances, of rotating about at least one axis or centre of rotation, the said application surface being in liquid communication with the said container via at least one passage which, over at least a portion of its length, exhibits a diameter of 1 to 6 mm, preferably of 2 to 5 mm and better still of 2 to 4 mm.

According to a preferred embodiment, the said passage emerges with regard to the said application surface via at least one orifice exhibiting a diameter of 1 to 6 mm, preferably of 2 to 5 mm and better still of 2 to 4 mm.

The term “diameter” is understood to mean in particular the diameter inscribed in the geometric shape of the orifice, for the case where the orifice has a noncircular shape.

According to a preferred form, the orifice has a circular shape.

This is because the inventors have observed that:
at low shear (from 10⁻³ to 10⁻¹ s⁻¹), a correlation may be made between the viscosity of the composition and the ability to start up the applicator: the lower the viscosity, the easier is the startup.
at high shear (1000 s⁻¹), a correlation may be made between the viscosity of the composition and the amount of composition released: the viscosity should be neither too high nor too low. Furthermore, the quality of the correlation may be made between the viscosity of the composition and the ability to apply this composition via the applicator.

The composition according to the invention can be a composition for making up and/or caring for the skin, in particular the face and/or body, and can constitute a blusher, an eyeshadow, a foundation, a concealer, a lipstick, a product for making up the body, a product for caring for the face or the body or an antisun product.

More especially but nonexclusively, the invention relates to a foundation composition.

The emulsion according to the invention has a specific rheological characteristic which renders its use to be particularly advantageous. Thus, during application to the keratinous substance, in particular the skin, under the pressure exerted by the applicator according to the invention, the emulsion liquefies under the effect of the shearing due to the movement of the application means over the skin.

It is said that the composition according to the invention has a shear-thinning nature or behaviour, that is to say that the viscosity of the composition can be reversibly lowered when increasing shears are applied to the composition.

Such a composition exhibits different levels of viscosity at low (10⁻² s⁻¹) and high (10³ s⁻¹) shear.

### Viscosity Measurements

The viscosity measurements are carried out using a controlled-stress rheometer, AR-G2, TA Instruments, equipped with a cone-plate measuring body provided with an antievaporation device (cover). The measurements are carried out at 25° C.

A measurement of viscosity is carried out at all the shear rates between 0.001 and 1000 s⁻¹ while conforming to the following rheological protocol:
Preshearing: shearing is carried out at 1000 s⁻¹ for 1 min, followed by a resting time of 10 min.
Controlled-rate shearing cycle: A logarithmic rising slope from 0.001 to 1000 s⁻¹ in 2 minutes, followed by shearing at 1000 s⁻¹ for 1 min and, finally, a logarithmic descending slope from 1000 to 0.001 s⁻¹ in 2 min is produced.

According to another aspect, the invention relates to a method for making up and/or caring for a keratinous substance, in particular the skin, in which an amount of a composition, in particular in the liquid foundation form, is withdrawn and applied simultaneously using an application device according to the invention comprising a container containing the said composition and an applicator head integral with the said container supporting an application means, the said application means comprising an application surface capable, in response to its movement on engaging with the keratinous substances, of rotating about an axis or centre of rotation, in particular as defined below, the said composition being in the water-in-oil emulsion form, characterized by the following rheological profile:
a viscosity of between 35 and 1000 Pa·s, in particular between 40 and 950 Pa·s, preferably between 55 and 900 Pa·s, at a shear gradient of 0.01 s⁻¹,
a viscosity of between 0.20 and 0.90 Pa·s, in particular between 0.25 and 0.80 Pa·s, preferably between 0.28 and 0.70 Pa·s, at a shear gradient of 1000 s⁻¹,
   as measured using a controlled-stress rheometer, AR-G2, TA Instruments, at 25° C., the said application surface being in liquid communication with the said container via at least one passage which, over at least a portion of its length, exhibits a diameter of 1 to 6 mm, preferably of 2 to 5 mm and better still of 2 to 4 mm.

According to a specific form, the application device is characterized in that the passage emerges with regard to the said application surface via at least one orifice exhibiting a diameter of 1 to 6 mm, preferably of 2 to 5 mm and better still of 2 to 4 mm.

### Application Device

The application device according to the invention comprises a container and an applicator head supporting an application means, the application means comprising an application surface capable, in response to its movement on engagement with the keratinous substances, in particular the skin, in rotating about at least one axis or centre of rotation.

Reference is made to application devices of “dispenser-applicator” type, such as those described in Application WO2006/090061.

During application, the application means can rotate without sliding over the skin. Several successive passes may be carried out at the same spot, according to, for example, the intensity of colour desired. During successive passes, the user can slightly modify the direction of movement, in order to soften the edges of the deposited layer of product.

Such an application device makes possible good control and is very easy to handle.

In addition, the use of application means in accordance with the invention to carry out the deposition of a composition at the surface of a keratinous substance and more particularly of the skin makes it possible to obtain a massaging effect on the skin, of the sort to provide pleasant sensory feelings for the user.

The method of application according to the invention furthermore exhibits the advantage of not sullying the fingers with the composition in the application.

Furthermore, the use of an application means according to the invention makes it possible to obtain a makeup which is significantly improved in terms of homogeneity of the deposited layer, providing a discreet and natural makeup effect.

The application means can be defined by a roller, a ball or a belt positioned around two rollers having parallel axes of rotation. Preferably, it is a roller, then distinct from a ball.

In a particularly preferred embodiment, the application surface of said applicator device is of the foam type, thereby different from plastic or metal materials generally used for rotating balls of roll-on dispensing systems, like roll-on deodorants and antiperspirants.

In particular, the use of a roller presenting a larger application surface in contact with the keratinous material makes it possible to improve the quality of the final makeup, especially in terms of uniformity of the deposit.

The application means is advantageously provided in the form of a hollow or solid roller which rotates about an axis of rotation. This axis of rotation can advantageously be positioned perpendicular to the longitudinal axis of the device.

The applicator head which carries the application means can be movable relative to the container, in particular rotary with respect to the latter, in particular about its longitudinal axis. The movement of the applicator head relative to the container can bring about that of a shutter, causing it to pass from a shutting position closing a communication between the orifice or orifices and the container to an open position, and vice versa (on/off position).

The applicator head can comprise two uprights between which the application means rotates.

The applicator head and/or the container can also comprise one or more channels making it possible to convey the product to the surface of the application means.

The application surface dedicated to contact with a keratinous substance can in particular be a foam comprising open or closed cells, which cells are optionally flocked, a flocking, an elastomer, a sintered material, a woven material or a nonwoven material. Preferably, it is a foam comprising open or closed cells.

The application surface may or may not be smooth. Thus, this application means can advantageously have raised areas at the surface, generally humped and rounded, advantageous in jointly providing a massaging effect.

In the device used according to the invention, the application surface is in liquid communication with the said container via at least one passage which, over at least a portion of its length, exhibits a diameter of 1 to 6 mm, preferably of 2 to 5 mm and better still of 2 to 4 mm.

According to a preferred embodiment, the passage emerges with regard to the said application surface via at least one orifice exhibiting a diameter of 1 to 6 mm, preferably of 2 to 5 mm and better still of 2 to 4 mm.

The composition stored in the container is thus delivered directly to the surface of the application means from the outside via at least one orifice exhibiting a diameter of 1 to 6 mm, preferably of 2 to 5 mm and better still of 2 to 4 mm. The impregnation of the application means with the composition and its movement over the keratinous substance are thus carried out simultaneously.

The container can comprise a variable volume in order to place the product under pressure and to force the dispensing thereof through the orifice or orifices.

According to a first embodiment, the container can comprise a flexible wall, it being possible for the user to press against this wall and thus to place the product under pressure.

According to an alternative embodiment, the container comprises a piston which the user can actuate in order to place the product under pressure and to force the dispensing thereof through the orifice or orifices.

The orifice can be in selective communication with the container: the passage can comprise a valve and/or a shutting system.

The shutter can be movable relative to the container between a shutting position, where it closes the communication between the orifice and the container, and a dispensing position, where it re-establishes the said communication.

### Examples of Cosmetic Packaging and Application Combinations for the Implementation of the Invention

An example among others of an application device of “dispenser-applicator” type which can be used according to the invention and which is particularly suited to the application of a liquid composition exhibiting the rheological profile according to the invention has been represented in the figures.

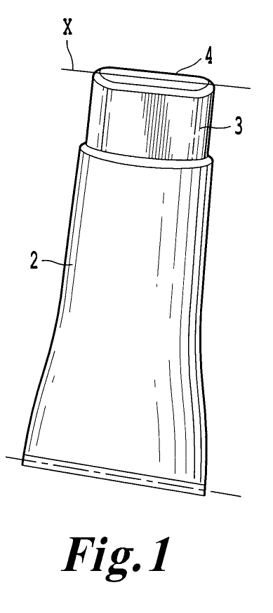

According to one embodiment, the packaging and application device 1 represented in FIG. 1 is provided in the form of a dispenser-applicator tube suitable for the storage, dispensing and application of a liquid product P which is more or less viscous.

Such applicators-dispensers are described in particular in Application WO 2006/090061.

Such a device can comprise a container for storage of the composition in the form of a flexible tube 2 comprising, at its end, an applicator head 3 supporting an application means 4.

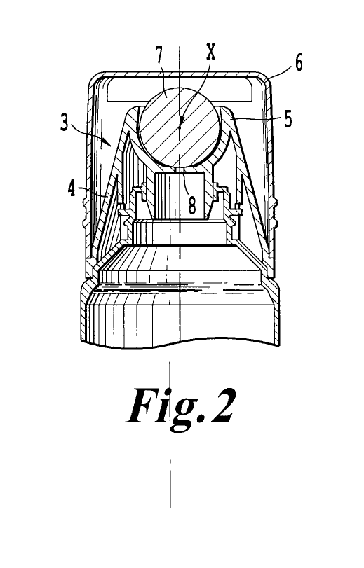

As represented in FIG. 2, the applicator head 3 is fitted to the neck of the tube via a fastening skirt 4. In addition, the head comprises a hollow support 5 carrying the application means. The head is fitted to the tube in leaktight fashion by virtue of a leaktightness skirt.

The flexible tube 2 can have any appropriate shape and be of different materials, in particular plastic or thermoplastic materials.

Advantageously, the head is surmounted by a suitable cap 6 which protects the application device during storage of the tube.

The application means 7 can be provided in the form of a roller, the application surface of which can be cylindrical with revolution about the X axis. This application means is capable of rotating and can withdraw, on one side, the product originating from the container while applying it on the other side.

The applicator roller 7 can thus rotate in the said hollow support, fed with product via an orifice 8 exhibiting a diameter of 3 mm, and can carry the product to the keratinous substance.

In order to use the packaging and application device represented in FIGS. 1 and 2, the user can, in order to feed the applicator head 3 with product, press against the flexible wall of the tube 2 while continuing to apply the said application means 7 to the keratinous substance to be treated, for example the skin.

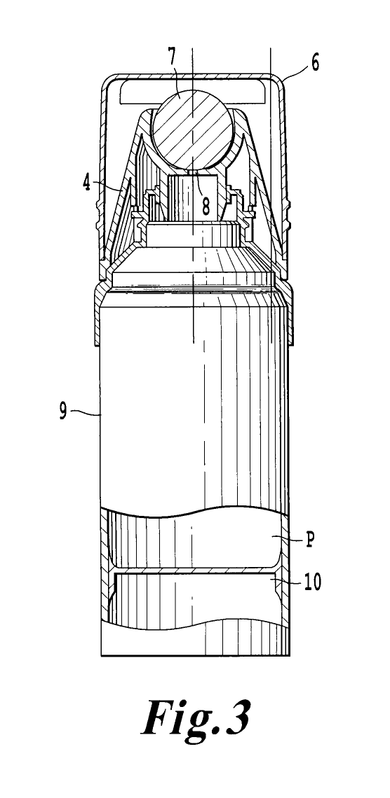

According to another alternative embodiment, the packaging and application combination represented in FIG. 3 can comprise a container 9 for storage of the product P in which can slide a piston 10, making it possible to force the product P towards the application means 7 fed via at least one internal channel 8.

### Composition

The composition according to the invention is in the form of a water-in-oil emulsion comprising an aqueous phase dispersed in a liquid fatty phase and comprises at least one physiologically acceptable medium.

The term “physiologically acceptable medium” is intended to denote a medium particularly suitable for the application of a composition of the invention to keratinous substances, in particular the skin and lips.

The physiologically acceptable medium is generally suited to the nature of the substrate on which the composition has to be applied and to the appearance under which the composition has to be packaged.

The said composition is characterized by the rheological profile defined above, namely:
a viscosity of between 35 and 1000 Pa·s, in particular between 40 and 950 Pa·s, preferably between 55 and 900 Pa·s, at a shear gradient of 0.01 s⁻¹,
a viscosity of between 0.20 and 0.90 Pa·s, in particular between 0.25 and 0.80 Pa·s, preferably between 0.28 and 0.70 Pa·s, at a shear gradient of 1000 s⁻¹,
   as measured using a controlled-stress rheometer, AR-G2, TA Instruments, at 25° C.

It is characterized in particular by an aqueous phase dispersed in a liquid fatty phase, a surfactant and the presence optionally of at least one agent intended to increase the viscosity of the said composition.

### Aqueous Phase

A composition according to the invention comprises at least one aqueous phase, preferably in a content ranging from 10 to 80% by weight and more particularly from 30 to 70% by weight, with respect to the total weight of the composition.

The aqueous phase comprises water and/or at least one water-soluble solvent.

The water can be a floral water, such as cornflower water, and/or a mineral water, such as water from Vittel, water from Lucas or water from La Roche-Posay, and/or thermal water.

The term “water-soluble solvent” denotes, in the present invention, a compound which is liquid at ambient temperature and which is miscible with water (miscibility in water of greater than 50% by weight at 25° C. and atmospheric pressure).

The water-soluble solvent(s) suitable for the invention can be chosen from C₁₋₈ monoalcohols, in particular C₁₋₅ monoalcohols, in particular ethanol, isopropanol, tert-butanol or n-butanol, polyols, such as described above, and their mixtures. Also very particularly suitable for the invention are ethanol and isopropanol and preferably ethanol.

A composition of the invention can additionally comprise at least one salt, for example sodium chloride, magnesium chloride and magnesium sulphate.

A composition of the invention can comprise from 0.05 to 1.5% by weight, in particular from 0.1 to 1.0% by weight and more particularly from 0.15 to 0.8% by weight of salts, with respect to the total weight of the composition.

The aqueous phase can also comprise any water-soluble or water-dispersible compound compatible with an aqueous phase, such as gelling agents, film-forming polymers, thickeners, surfactants and their mixtures.

### Liquid Fatty Phase

A cosmetic composition in accordance with the present invention comprises at least one liquid fatty phase and in particular at least one oil as mentioned below.

The term “oil” is understood to mean any fatty substance in the liquid form at ambient temperature (20-25° C.) and at atmospheric pressure.

A composition of the invention can comprise a liquid fatty phase in a content varying from 10 to 90% by weight, in particular from 15 to 60% by weight and more particularly from 20 to 50% by weight, with respect to the total weight of the composition.

A composition according to the invention can comprise at least one oil chosen from volatile and nonvolatile oils of hydrocarbon, silicone or fluorinated type. The oils can be of animal, vegetable, mineral or synthetic origin.

The term “oil” is understood to mean any fatty substance in the liquid form at ambient temperature (20-25° C.) and at atmospheric pressure.

The term “volatile oil” is understood to mean an oil (or nonaqueous medium) capable of evaporating on contact with the skin in less than one hour at ambient temperature and atmospheric pressure. The volatile oil is a volatile cosmetic oil which is liquid at ambient temperature and which has in particular a nonzero vapour pressure at ambient temperature and atmospheric pressure, in particular which has a vapour pressure ranging from 0.13 Pa to 40 000 Pa (10⁻³ to 300 mmHg), preferably ranging from 1.3 Pa to 13 000 Pa (0.01 to 100 mmHg) and preferentially ranging from 1.3 Pa to 1300 Pa (0.01 to 10 mmHg).

In addition, the volatile oil generally has a boiling point, measured at atmospheric pressure, ranging from 150° C. to 260° C. and preferably ranging from 170° C. to 250° C.

Advantageously, the fatty phase comprises from 5 to 40% by weight, preferably ranging from 10 to 35% by weight and preferentially ranging from 15 to 30% by weight of volatile oil(s), with respect to the total weight of the composition.

The term “hydrocarbon oil” is understood to mean an oil essentially formed, indeed even composed, of carbon and hydrogen atoms and optionally of oxygen and nitrogen atoms which does not comprise a silicon or fluorine atom; it can comprise ester, ether, amine and amide groups.

The term “silicone oil” is understood to mean an oil comprising at least one silicon atom and in particular comprising Si—O groups.

The term “fluorinated oil” is understood to mean an oil comprising at least one fluorine atom.

### Volatile Oils

The volatile hydrocarbon oil which can be used in the invention can be chosen from hydrocarbon oils having a flash point ranging from 40° C. to 102° C., preferably ranging from 40° C. to 55° C. and preferably ranging from 40° C. to 50° C.

Mention may be made, as volatile hydrocarbon oil, of volatile hydrocarbon oils having from 8 to 16 carbon atoms and their mixtures, in particular branched C₈-C₁₆ alkanes, such as C₈-C₁₆ isoalkanes (also known as isoparaffins), isododecane, isodecane, isohexadecane and, for example, the oils sold under the Isopar or Permethyl commercial names, branched C₈-C₁₆ esters, such as isohexyl neopentanoate, and their mixtures. Preferably, the volatile hydrocarbon oil is chosen from volatile hydrocarbon oils having from 8 to 16 carbon atoms and their mixtures, in particular from isododecane, isodecane or isohexadecane, and is in particular isododecane.

The volatile hydrocarbon oil can be present in a content ranging from 5 to 20% by weight, with respect to the total weight of the emulsion, preferably ranging from 6 to 15% by weight and preferentially ranging from 7 to 12% by weight.

The volatile silicone oil which can be used in the invention can be chosen from silicone oils having a flash point ranging from 40° C. to 102° C., preferably having a flash point of greater than 55° C. and less than or equal to 95° C., and preferentially ranging from 65° C. to 95° C.

Mention may be made, as volatile silicone oil, of linear or cyclic silicone oils having from 2 to 7 silicone atoms, these silicones optionally comprising alkyl or alkoxy groups having from 1 to 10 carbon atoms. Mention may in particular be made, as examples of volatile silicone oil, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and their mixtures.

Mention may be made, as volatile fluorinated oil, of nonafluoroethoxybutane, nonafluoromethoxybutane, decafluoropentane, tetradecafluorohexane, dodecafluoropentane and their mixtures.

The volatile oil chosen from volatile silicone oils, volatile fluorinated oils and their mixtures can be present in a content ranging from 0 to 32% by weight, with respect to the total weight of the emulsion, preferably ranging from 10 to 30% by weight and preferentially ranging from 15 to 25% by weight, with respect to the total weight of the composition.

The fatty phase of the emulsion according to the invention can additionally comprise at least one nonvolatile oil.

### Nonvolatile Oils

This nonvolatile oil or one of its mixtures can be present in a content ranging from 0.1 to 40% by weight, with respect to the total weight of the emulsion, and preferably ranging from 1 to 25% by weight.

The nonvolatile oil can be chosen from carbon-comprising, hydrocarbon-comprising and/or silicone oils of mineral, animal, vegetable or synthetic origin, and their mixtures, in so far as they are compatible with the use envisaged.

Mention may be made of nonvolatile hydrocarbon oils, such as liquid paraffin or liquid petrolatum, isoeicosane, soybean oil, perhydrosqualene, sweet almond, calophyllum, palm, grape seed, sesame, maize, arara, rapeseed, sunflower, cottonseed, apricot, castor, avocado, jojoba, olive or cereal germ oil; esters of lanolic acid, oleic acid, lauric acid or stearic acid; fatty esters, such as isopropyl myristate, isopropyl palmitate, butyl stearate, hexyl laurate, diisopropyl adipate, isononyl isononanoate, 2-ethylhexyl palmitate, 2-hexyldecyl laurate, 2-octyldecyl palmitate, 2-octyldodecyl myristate or lactate, di(2-ethylhexyl)succinate, diisostearyl malate, glyceryl triisostearate or diglyceryl triisostearate; higher fatty acids, such as myristic acid, palmitic acid, stearic acid, behenic acid, oleic acid, linoleic acid, linolenic acid or isostearic acid; or higher fatty alcohols, such as cetanol, stearyl alcohol or oleyl alcohol, linoleyl or linolenyl alcohol, isostearyl alcohol or octyldodecanol.

Mention may be made, as nonvolatile silicone oil, of polydimethylsiloxanes (PDMSs), which are optionally phenylated, such as phenyl trimethicones, or optionally substituted by aliphatic and/or aromatic groups or by functional groups, such as hydroxyl, thiol and/or amine groups; polysiloxanes modified with fatty acids, fatty alcohols or polyoxyalkylenes, and their mixtures.

### Surface-Active Agents

A composition according to the invention comprises one or more surface-active agent(s) suitable for W/O emulsions chosen in particular from amphoteric, anionic, cationic or nonionic surface-active agents, used alone or as mixtures.

The surface-active agents are generally present in the composition in a content which can range from 0.5 to 15% by weight, in particular from 1.5 to 5% by weight, with respect to the total weight of the composition.

By way of example, the silicone surfactant(s) can be present in a content ranging from 0.5 to 10% by weight, in particular from 1 to 5% by weight, with respect to the total weight of the composition.

According to another specific embodiment, the nonsilicone surfactant(s) can be present in a content ranging from 1 to 10% by weight, in particular from 2 to 8% by weight, with respect to the total weight of the composition.

For the W/O emulsions, use may in particular be made of hydrocarbon-comprising or silicone surface-active agents.

Mention may be made, for example, as hydrocarbon-comprising surface-active agents, of polyesters of polyols, such as PEG-30 dipolyhydroxystearate, sold under the reference Arlacel P 135 by Uniqema, or polyglyceryl-2 dipolyhydroxystearate, sold under the reference Dehymuls PGPH by Cognis.

Mention may be made, for example, as silicone surface-active agents, of alkyl dimethicone copolyols, such as lauryl dimethicone copolyol, sold under the name “Dow Corning 5200 Formulation Aid” by Dow Corning, and cetyl dimethicone copolyol, sold under the name Abil EM 90 by Goldschmidt, or the polyglyceryl-4 isostearate/cetyl dimethicone copolyol/hexyl laurate mixture sold under the name Abil WE 09 by Goldschmidt.

One or more coemulsifiers can also be added thereto. Advantageously, the coemulsifier can be chosen from the group consisting of polyol alkyl esters. Mention may in particular be made, as polyol alkyl esters, of esters of glycerol and/or of sorbitan, for example polyglyceryl-3 diisostearate, sold under the name of Lameform TGI by Cognis, polyglycerol-4 isostearate, such as the product sold under the name Isolan GI 34 by Goldschmidt, sorbitan isostearate, such as the product sold under the name Arlacel 987 by ICI, glycerol sorbitan isostearate, such as the product sold under the name Arlacel 986 by ICI, and their mixtures.

According to a particularly preferred embodiment, an emulsion according to the invention, in particular a W/O emulsion comprising a silicone oily phase, comprises at least one silicone surfactant more particularly chosen from dimethicone copolyols.

The presence of a dimethicone copolyol promotes in particular the stabilization of the emulsion according to the invention.

A dimethicone copolyol which can be used according to the invention is an oxypropylenated and/or oxyethylenated polydimethylmethylsiloxane.

Use may be made, as dimethicone copolyol, of those corresponding to the following formula (II):

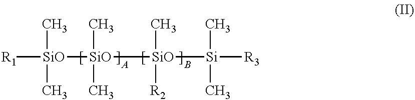

in which:
R₁, R₂ and R₃ represent, independently of one another, a C₁-C₆ alkyl radical or a —(CH₂)ₓ—(OCH₂CH₂)_{y}—(OCH₂CH₂CH₂)_{z}—OR₄ radical, at least one R₁, R₂ or R₃ radical not being an alkyl radical; and R₄ being a hydrogen, a C₁-C₃ alkyl radical or a C₂-C₄ acyl radical;
A is an integer ranging from 0 to 200;
B is an integer ranging from 0 to 50; provided that A and B are not equal to zero at the same time;
x is an integer ranging from 1 to 6;
y is an integer ranging from 1 to 30; and
z is an integer ranging from 0 to 5.

According to a preferred embodiment of the invention, in the compound of formula (II), R₁=R₃=methyl radical, x is an integer ranging from 2 to 6 and y is an integer ranging from 4 to 30. R₄ is in particular a hydrogen.

Mention may be made, as examples of compounds of formula (II), of the compounds of formula (III):

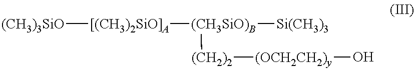

in which A is an integer ranging from 20 to 105, B is an integer ranging from 2 to 10 and y is an integer ranging from 10 to 20.

Mention may also be made, as examples of silicone compounds of formula (II), of the compounds of formula (IV):

HO—(OCH₂CH₂)_{y}—(CH₂)₃—[(CH₃)₂SiO]_{A′}—(CH₂)₃—(OCH₂CH₂)_{y}—OH (IV)

in which A′ and y are integers ranging from 10 to 20.

Use may be made, as dimethicone copolyols, of those sold under the names DC 5329, DC 7439-146, DC-2-5695 and Q4-3667 by Dow Corning and KF-6013, KF-6015, KF-6016, KF-6017 and KF-6028 by Shin-Etsu.

The compounds DC 5329, DC 7439-146 and DC 2-5695 are compounds of formula (III) where, respectively, A is 22, B is 2 and y is 12, A is 103, B is 10 and y is 12, and A is 27, B is 3 and y is 12.

According to a specific embodiment, the silicone surfactant can be PEG-9 polydimethylsiloxyethyl dimethicone, sold in particular by Shin-Etsu under the reference KF-6028, PEG-10 dimethicone, sold in particular by Shin-Etsu under the reference KF-6017, and their mixtures.

The dimethicone copolyol can be present in the composition according to the invention in a content ranging from 1 to 6% by weight, with respect to the total weight of the emulsion, preferably ranging from 1.5 to 4% by weight and preferentially ranging from 2 to 3% by weight.

According to a preferred alternative embodiment of the invention, the abovementioned dimethicone copolyol can be combined with at least one α,ω-substituted oxyalkylenated silicone.

α,ω-Substituted Oxyalkylenated Silicone

In everything which follows or which precedes, the term “silicone” is understood to denote, in conformity with what is generally accepted, any organosilicon polymer or oligomer with a branched or crosslinked and linear or cyclic structure of variable molecular weight obtained by polymerization and/or polycondensation of suitably functionalized silanes and essentially composed of a repetition of main units in which the silicon atoms are connected to one another via oxygen atoms (siloxane bond ≡Si—O—Si≡), optionally substituted hydrocarbon radicals being directly bonded via a carbon atom to the said silicon atoms. The commonest hydrocarbon radicals are alkyl radicals, in particular C₁-C₁₀ alkyl radicals and especially methyl radicals, fluoroalkyl radicals and aryl radicals, in particular phenyl radicals. They can, for example, be substituted by C₁-C₄₀ ester or ether groups or C₇-C₆₀ aralkyl groups.

Thus, the α,ω-substituted oxyalkylene silicone which can be used according to the invention is an organosilicon polymer as defined above with a linear structure which is substituted at the two ends of the main chain by oxyalkylene groups connected to the Si atoms via a hydrocarbon group.

Preferably, the α,ω-substituted oxyalkylenated silicone corresponds to the following general formula (I):

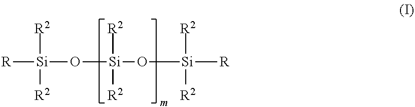

in which: R═—(CH₂)ₚO—(C₂H₄O)ₓ(C₃H₆O)_{Y}R¹
where: R¹ represents H, CH₃ or CH₂CH₃,
   p is an integer ranging from 1 to 5, x varies from 1 to 100 and y varies from 0 to 50,
   it being possible for the (C₂H₄O) and (C₃H₆O) units to be distributed randomly or in blocks,
   the R² radicals represent a C₁-C₃ alkyl radical or a phenyl radical, and
   5≦m≦300.

Preferably, the α,ω-substituted oxyalkylenated silicone used according to the present invention corresponds to the general formula (I) for which all the R² radicals are methyl radicals and:
p ranges from 2 to 4,
x ranges from 3 to 100,
m ranges from 50 to 200.

Preferably again, the average molecular weight of R ranges from 800 to 2600.

Preferably, the ratio by weight of the C₂H₄O units with respect to the C₃H₆O units ranges from 100:10 to 20:80. Advantageously, this ratio is approximately 42/58.

Preferably again, R¹ is the methyl group.

More preferably still, the emulsion according to the invention comprises the α,ω-oxyalkylenated silicone of following formula:

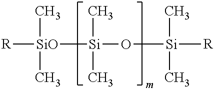

in which:
m=100,
R═(CH₂)₃—O—(C₂H₄O)ₓ—(C₃H₆O)_{y}—CH₃, where x ranges from 3 to 100 and y ranges from 1 to 50, the ratio by weight of the number of C₂H₄O to the number of C₃H₆O being approximately 42/58 and the average molecular weight of R ranging from 800 to 1000.

The α,ω-substituted oxyalkylenated silicone as defined above can be used according to the invention in a proportion ranging from 0.5 to 5% by weight, in particular from 1 to 4% by weight and more particularly from 2 to 3% by weight, with respect to the total weight of the composition.

Mention may in particular be made, among the commercial products which may comprise all or part of the α,ω-substituted oxyalkylenated silicones which can be used according to the invention as emulsifier, of those sold under the names of “Abil EM 97” by Goldschmidt or also of “KF 6009”, “X22-4350”, “X22-4349” or “KF 6008” by Shin Etsu.

In particular, the product can be cetyl dimethicone copolyol.

Emulsifying Silicone Elastomers

Use may also be made, as surface-active agents for W/O emulsions, of emulsifying silicone elastomer.

Advantageously, a composition according to the invention comprises at least one emulsifying silicone elastomer, optionally in combination with a dimethicone copolyol as described above.

The emulsifying silicone elastomer can be chosen from a polyoxyalkylenated silicone elastomer, a polyglycerolated silicone elastomer and their mixtures.

Use will preferably be made of a polyglycerolated silicone elastomer.

The polyoxyalkylenated silicone elastomer is a crosslinked organopolysiloxane which can be obtained by a crosslinking addition reaction of a diorganopolysiloxane comprising at least one hydrogen bonded to silicon and of a polyoxyalkylene having at least two groups comprising ethylenic unsaturation.

Preferably, the polyoxyalkylenated crosslinked organopolysiloxane is obtained by a crosslinking addition reaction (A1) of a diorganopolysiloxane comprising at least two hydrogens each bonded to a silicon and (B1) of a polyoxyalkylene having at least two groups possessing ethylenic unsaturation, in particular in the presence (C1) of a platinum catalyst, such as, for example, described in Patents U.S. Pat. No. 5,236,986 and U.S. Pat. No. 5,412,004.

In particular, the organopolysiloxane can be obtained by reaction of a polyoxyalkylene (in particular a polyoxyethylene and/or polyoxypropylene) possessing dimethylvinylsiloxy ends and of a methylhydropolysiloxane possessing trimethylsiloxy ends, in the presence of a platinum catalyst.

The organic groups bonded to the silicon atoms of the compound (A1) can be alkyl groups having from 1 to 18 carbon atoms, such as methyl, ethyl, propyl, butyl, octyl, decyl, dodecyl (or lauryl), myristyl, cetyl or stearyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbon groups, such as an epoxy group, a carboxylate ester group or a mercapto group.

The compound (A1) can thus be chosen from methylhydropolysiloxanes possessing trimethylsiloxy ends, dimethylsiloxane/methylhydrosiloxane copolymers possessing trimethylsiloxy ends, cyclic dimethylsiloxane/methylhydrosiloxane copolymers, or dimethylsiloxane/methylhydrosiloxane/laurylmethylsiloxane copolymers possessing trimethylsiloxy ends.

The compound (C1) is the catalyst of the crossslinking reaction and is in particular chloroplatinic acid, chloroplatinic acid/olefin complexes, chloroplatinic acid/alkenylsiloxane complexes, chloroplatinic acid/diketone complexes, platinum black and platinum-on-support.

Advantageously, the polyoxyalkylenated silicone elastomers can be formed from divinyl compounds, in particular polyoxyalkylenes having at least two vinyl groups, which react with Si—H bonds of a polysiloxane.

The polyoxyalkylenated silicone elastomer according to the invention is preferably mixed with at least one hydrocarbon oil and/or one silicone oil in order to form a gel. In these gels, the polyoxyalkylenated elastomer is in the form of nonspherical particles.

Polyoxyalkylenated elastomers are described in particular in Patents U.S. Pat. No. 5,236,986, U.S. Pat. No. 5,412,004, U.S. Pat. No. 5,837,793 and U.S. Pat. No. 5,811,487.

Use can be made, as polyoxyalkylenated silicone elastomer, of those sold under the names “KSG-21”, “KSG-20”, “KSG-30”, “KSG-31”, KSG-32”, “KSG-33”, “KSG-210”, “KSG-310”, “KSG-320”, “KSG-330”, “KSG-340” and “X-226146” by Shin Etsu and “DC9010” and “DC9011” by Dow Corning.

According to another embodiment, the emulsifying silicone elastomer can advantageously be chosen from polyglycerolated silicone elastomers.

The polyglycerolated silicone elastomer is a crosslinked organopolysiloxane elastomer which can be obtained by a crosslinking addition reaction of a diorganopolysiloxane comprising at least one hydrogen bonded to silicon and of polyglycerolated compounds having groups possessing ethylenic unsaturation, in particular in the presence of a platinum catalyst.

Preferably, the crosslinked organopolysiloxane elastomer is obtained by a crosslinking addition reaction (A) of a diorganopolysiloxane comprising at least two hydrogens each bonded to a silicon and (B) of glycerolated compounds having at least two groups possessing ethylenic unsaturation, in particular in the presence (C) of a platinum catalyst.

In particular, the organopolysiloxane can be obtained by reaction of a polyglycerolated compound possessing dimethylvinylsiloxy ends and of a methylhydropolysiloxane possessing trimethylsiloxy ends, in the presence of a platinum catalyst.

The compound (A) is the base reactant for the formation of an organopolysiloxane elastomer and the crosslinking takes place by an addition reaction of the compound (A) with the compound (B) in the presence of the catalyst (C).

The compound (A) is in particular an organopolysiloxane having at least 2 hydrogen atoms bonded to separate silicon atoms in each molecule.

The compound (A) can exhibit any molecular structure, in particular a linear chain or branched chain structure or a cyclic structure.

The compound (A) can have a viscosity at 25° C. ranging from 1 to 50 000 centistokes, in particular in order to be satisfactorily miscible with the compound (B).

The organic groups bonded to the silicon atoms of the compound (A) can be alkyl groups having from 1 to 18 carbon atoms, such as methyl, ethyl, propyl, butyl, octyl, decyl, dodecyl (or lauryl), myristyl, cetyl or stearyl; substituted alkyl groups, such as 2-phenylethyl, 2-phenylpropyl or 3,3,3-trifluoropropyl; aryl groups, such as phenyl, tolyl or xylyl; substituted aryl groups, such as phenylethyl; and substituted monovalent hydrocarbon groups, such as an epoxy group, a carboxylate ester group or a mercapto group. Preferably, the said organic group is chosen from the methyl, phenyl and lauryl groups.

The compound (A) can thus be chosen from methylhydropolysiloxanes possessing trimethylsiloxy ends, dimethylsiloxane/methylhydrosiloxane copolymers possessing trimethylsiloxy ends, cyclic dimethylsiloxane/methylhydrosiloxane copolymers, or dimethylsiloxane/methylhydrosiloxane/laurylmethylsiloxane copolymers possessing trimethylsiloxy ends.

The compound (B) can be a polyglycerolated compound corresponding to the following formula (B′):

CₘH₂ₘ₋₁—O-[Gly]ₙ-CₘH₂ₘ₋₁ (B′)

in which m is an integer ranging from 2 to 6, n is an integer ranging from 2 to 200, preferably ranging from 2 to 100, preferably ranging from 2 to 50, preferably ranging from 2 to 20, preferably ranging from 2 to 10 and preferentially ranging from 2 to 5 and in particular equal to 3; Gly denotes:

—CH₂—CH(OH)—CH₂—O— or —CH₂—CH(CH₂OH)—O—

Advantageously, the sum of the number of ethylene groups per molecule of the compound (B) and of the number of hydrogen atoms bonded to silicon atoms per molecule of the compound (A) is at least 4.

It is advantageous for the compound (A) to be added in an amount such that the molecular ratio of the total amount of hydrogen atoms bonded to silicon atoms in the compound (A) to the total amount of all the groups possessing ethylenic unsaturation in the compound (B) is within the range from 1/1 to 20/1.

The compound (C) is the catalyst of the crosslinking reaction and is in particular chloroplatinic acid, chloroplatinic acid/olefin complexes, chloroplatinic acid/alkenylsiloxane complexes, chloroplatinic acid/diketone complexes, platinum black and platinum-on-support.

The catalyst (C) is preferably added from 0.1 to 1000 parts by weight, better still from 1 to 100 parts by weight, as platinum metal proper, per 1000 parts by weight of the total amount of the compounds (A) and (B)

The polyglycerolated silicone elastomer according to the invention is generally mixed with at least one hydrocarbon oil and/or one silicone oil in order to form a gel. In these gels, the polyglycerolated elastomer is often in the form of nonspherical particles.

Such elastomers are described in particular in Patent Application WO 2004/024798.

Use may be made, as polyglycerolated silicone elastomers, of those sold under the names “KSG-710”, “KSG-810”, “KSG-820”, “KSG-830” and “KSG-840” by Shin Etsu.

According to a preferred form, use is made of a polyglycerolated emulsifying silicone elastomer at 25% by weight in polydimethylsiloxane (6 cSt), sold in particular under the name KSG-710 by Shin Etsu.

According to one aspect of the invention, these silicone elastomers can be present in the composition according to the invention in a total content as active material at least greater than 0.8% by weight, with respect to the total weight of the composition, in particular ranging from 1 to 4% by weight, preferably greater than or equal to 1.5% by weight, in particular ranging from 1.5 to 2.5% by weight, and more preferably greater than or equal to 1.6% by weight, in particular ranging from 1.6 to 2% by weight.

A composition according to the invention thus comprises, as fundamental architecture, an aqueous phase dispersed in an oily phase and at least one surfactant chosen from silicone or hydrocarbon-comprising surfactants.

Depending on the viscosity of the composition desired in order to be within the rheological profile according to the invention, a person skilled in the art can adjust the contents of aqueous phase, oily phase and/or surfactants.

He can also advantageously incorporate, in this fundamental architecture, one or more ingredients intended to adjust, in particular increase, the viscosity of the said composition. In particular, the composition according to the invention comprises at least one agent which increases the viscosity of the said composition.

Mention may in particular be made, as agents capable of increasing the viscosity of a composition, of inorganic or organic (in particular polymeric) lipophilic gelling agents, lipophilic structuring agents, particulate materials chosen from fillers, pigments or pearlescent agents, and their mixtures. These additional ingredients may in addition contribute cosmetic properties of texture, comfort, hold and possibly colour desired in the care and/or makeup field.

Thus, a composition of the invention can advantageously comprise at least one agent chosen from inorganic or organic (in particular polymeric) lipophilic gelling agents, lipophilic structuring agents, particulate materials chosen from fillers, pigments or pearlescent agents, and their mixtures.

According to a specific form of the invention, the composition comprises at least one lipophilic gelling agent.

### Lipophilic Gelling Agents

A gelling agent suitable for the invention is advantageously lipophilic. A lipophilic gelling agent can be inorganic or organic (in particular polymeric).

The lipophilic gelling agent or agents can be present in the composition in a content ranging from 0.1 to 20% by weight, in particular from 0.2 to 15% by weight and better still from 0.25 to 10% by weight, with respect to the total weight of the said composition.

### Inorganic Gelling Agents

Mention may be made, as lipophilic gelling agents, for example, of hydrophobic modified clays, such as modified magnesium silicate (Bentone Gel VS38 from Rheox), or hectorite modified by distearyldimethylammonium chloride (CTFA name: Disteardimonium hectorite), sold under the name “Bentone 38 CE” by Rheox.

Mention may be made, as inorganic lipophilic gelling agent, of optionally modified clays, such as hectorites modified by a C₁₀ to C₂₂ fatty acid ammonium chloride, such as hectorite modified by distearyldimethylammonium chloride, such as, for example, that sold under the name of Bentone 38V® by Elementis.

### Polymeric Organic Gelling Agents

The polymeric organic lipophilic gelling gents are, for example, partially or completely crosslinked organopolysiloxane elastomers, of three-dimensional structure, such as those sold under the names of KSG6®, KSG16® and KSG18® by Shin Etsu, of Trefil E-505C® and Trefil E-506C® by Dow Corning, of Gransil SR-CYC®, SR DMF10®, SR-DC556®, SR 5CYC Gel®, SR DMF 10 Gel® and SR DC 556 Gel® by Grant Industries, or of SF 1204® and JK 113® by General Electric; ethylcellulose, such as that sold under the name Ethocel® by Dow Chemical; polycondensates of polyamide type resulting from the condensation between a dicaboxylic acid comprising at least 32 carbon atoms and an alkylenediamine, in particular ethylenediamine, in which the polymer comprises at least one end carboxylic acid group esterified or amidated with at least one monoalcohol or one monoamine comprising from 12 to 30 carbon atoms which are linear and saturated, in particular ethylenediamine/stearyl dilinoleate copolymers, such as that sold under the name Uniclear 100 VG® by Arizona Chemical; galactomannans comprising from 1 to 6 and in particular from 2 to 4 hydroxyl groups per monosaccharide which are substituted by a saturated or unsaturated alkyl chain, such as guar gum alkylated by C₁ to C₆ and in particular C₁ to C₃ alkyl chains, and their mixtures.

Mention may also be made, as lipophilic gelling agent suitable for the invention, of copolymers of the polystyrene/polyalkylene type and more particularly block copolymers of “diblock”, “triblock” or “radial” type of the polystyrene/polyisoprene or polystyrene/polybutadiene type, such as those sold under the name Luvitol HSB® by BASF, of the polystyrene/copoly(ethylene-propylene) type, such as those sold under the name of Kraton® by Kraton Polymers, or of the polystyrene/copoly(ethylene-butylene) type, or blends of triblock and radial (star) copolymers in isododecane, such as those sold by Penreco under the name Versagel®, such as, for example, the blend of butylene/ethylene/styrene triblock copolymer and of ethylene/propylene/styrene star copolymer in isododecane (Versagel M 5960).

Mention may also be made, among the lipophilic gelling agents which can be used in a cosmetic composition of the invention, of esters of dextrin and of fatty acid, such as dextrin palmitates, in particular such as those sold under the names Rheopearl TL® or Rheopearl KL® by Chiba Flour, hydrogenated vegetable oils, such as hydrogenated castor oil, or fatty alcohols, in particular C₈ to C₂₆ and more particularly C₁₂ to C₂₂ fatty alcohols, such as, for example, myristyl alcohol, cetyl alcohol, stearyl alcohol or behenyl alcohol.

According to a particularly preferred embodiment, a composition according to the invention can comprise at least one inorganic lipophilic gelling agent chosen from hydrophobic modified clays.

According to a particularly preferred embodiment, a composition according to the invention can comprise at least one polymeric organic lipophilic gelling agent chosen in particular from copolymers of the polystyrene/polyalkylene type and more particularly copolymers of the polystyrene/copoly(ethylene-propylene) type, especially those sold under the name Kraton® by Kraton Polymers.

### Lipophilic Structuring Agents

The fatty phase can also comprise at least one lipophilic structuring agent.

The lipophilic structuring agent or agents can be present in the composition in a content ranging from 0.05 to 10% by weight, in particular from 0.1 to 8% by weight and better still from 0.2 to 5% by weight, with respect to the total weight of the said composition.

In particular, the said lipophilic structuring agent can be chosen from at least one wax, at least one gum and/or at least one pasty fatty substance of vegetable, animal, mineral or synthetic origin, indeed even silicone origin, and their mixtures.

### Waxes

Mention may be made, among waxes which are solid at ambient temperature and which are capable of being present in the composition according to the invention, of hydrocarbon waxes, such as beeswax, carnauba, candelilla, ouricury or Japan wax, cork fibre or sugarcane waxes, paraffin or lignite waxes, micro-crystalline waxes, lanolin wax, montan wax, ozokerites, polyethylene waxes, the waxes obtained by the Fischer-Tropsch synthesis, hydrogenated oils, or fatty esters and glycerides which are solid at 25° C. Use may also be made of silicone waxes, among which may be mentioned alkylpolymethylsiloxanes, alkoxypolymethylsiloxanes and/or polymethylsiloxane esters. The waxes can be provided in the form of stable dispersions of colloidal wax particles, such as can be prepared according to known methods, such as those of “Microemulsions Theory and Practice”, edited by L. M. Prince, Academic Press (1977), pages 21-32. Mention may be made, as wax which is liquid at ambient temperature, of jojoba oil.

Mention may also be made of hydrogenated vegetable oils, such as hydrogenated castor oil.

Mention may be made, as lipophilic structuring agent also suitable for the invention, of fatty alcohols, in particular C₈ to C₂₆ and more particularly C₁₂ to C₂₂ fatty alcohols.

According to one embodiment, a fatty alcohol suitable for the invention can be chosen from myristyl alcohol, cetyl alcohol, stearyl alcohol or behenyl alcohol.

Mention may be made, as lipophilic structuring agent also suitable for the invention, of esters of fatty acid and glycerols, such as glyceryl tristearate.

The waxes can be present in a proportion of 0.1 to 10% by weight, with respect to the total weight of the emulsion, preferably of 0.1 to 5% by weight.

### Pasty Fatty Compounds

Pasty fatty compounds can be defined using at least one of the following physicochemical properties:

a viscosity of 0.1 to 40 Pa·s (1 to 400 poises), preferably 0.5 to 25 Pa·s, measured at 40° C. with a Contraves TV rotary viscometer equipped with an MS-r3 or MS-r4 spindle at a frequency of 60 Hz,

a melting point of 25-70° C., preferably 25-55° C.

The compositions of the invention can also comprise at least one alkyl, alkoxy or phenyl dimethicone, such as, for example, the product sold under the name of “Abil Wax 2440” by Goldschmidt.

The composition according to the invention can comprise one or more particulate material(s) chosen from fillers, pulverulent colouring materials and their mixtures, constituting the pulverulent phase of the said composition.

### Pulverulent Phase

Within the meaning of the present invention, the term “pulverulent phase” covers all particles of the type of colouring materials and/or fillers, as defined below.

In particular, the composition comprises at least one particulate material chosen from pigments, fillers and their mixtures, in particular pigments.

According to a specific embodiment, a composition according to the invention can comprise at least 3% by weight, in particular more than 10% by weight, indeed even more than 12% by weight, of a pulverulent phase. The latter is dispersed therein in a homogeneous and stabilized form.

According to one embodiment, the composition of the invention comprises at least one filler.

### Fillers

A composition in accordance with the invention can also comprise at least one filler of organic or inorganic nature.

The term “filler” should be understood as meaning colourless or white solid particles of any shape which are provided in a form insoluble and dispersed in the medium of the composition. Of inorganic or organic nature, they make it possible to confer, on the composition, softness, mattness and uniformity on the makeup.

The fillers can be present in the emulsion in a content ranging from 0.1 to 10% by weight, with respect to the total weight of the emulsion, preferably from 0.5 to 7% by weight.

The fillers used in the compositions according to the present invention can be lamellar, globular or spherical in form, in the form of fibres or in any other form intermediary between these defined forms.

The fillers according to the invention may or may not be surface coated and, in particular, they may be treated at the surface with silicones, amino acids, fluorinated derivatives or any other substance promoting the dispersion and the compatibility of the filler in the composition.

Mention may be made, among inorganic fillers which can be used in the compositions according to the invention, of talc, mica, silica, trimethylsiloxysilicate, kaolin, bentone, calcium carbonate and magnesium hydrogencarbonate, hydroxyapatite, boron nitride, hollow silica microspheres (Silica Beads from Maprecos), glass or ceramic microcapsules, silica-based fillers, such as Aerosil 200 or Aerosil 300; Sunsphere H-33 or Sunsphere H-51, sold by Asahi Glass; Chemicelen, sold by Asahi Chemical; composites of silica and of titanium dioxide, such as the TSG series sold by Nippon Sheet Glass, and their mixtures.

Mention may be made, among the organic fillers which can be used in the compositions according to the invention, of powders formed of polyamide (Nylon® Orgasol from Atochem), of poly-β-alanine and of polyethylene, powders formed of polytetrafluoroethylene (Teflon®), lauroyllysine, starch, powders formed of tetrafluoroethylene polymers, hollow microspheres of polymers, such as Expancel (Nobel Industrie), metal soaps derived from organic carboxylic acids having from 8 to 22 carbon atoms, preferably from 12 to 18 carbon atoms, for example zinc stearate, magnesium stearate, lithium stearate, zinc laurate or magnesium myristate, Polypore® L 200 (Chemdal Corporation), silicone resin microbeads (Tospearl® from Toshiba, for example), polyurethane powders, in particular powders formed of crosslinked polyurethane comprising a copolymer, the said copolymer comprising trimethylol hexyllactone, such as the hexamethylene diisocyanate/trimethylol hexyllactone polymer sold under the name of Plastic Powder D-400® or Plastic Powder D-800® by Toshiki, carnauba microwaxes, such as that sold under the name of MicroCare 350® by Micro Powders, synthetic wax microwaxes, such as that sold under the name of MicroEase 114S® by Micro Powders, the microwaxes composed of a mixture of carnauba wax and of polyethylene wax, such as those sold under the names of MicroCare 300® and 310® by Micro Powders, the microwaxes composed of a mixture of carnauba wax and of synthetic wax, such as that sold under the name MicroCare 325® by Micro Powders, polyethylene microwaxes, such as those sold under the names of Micropoly 200®, 220®, 220L® and 250S® by Micro Powders, or fibres of synthetic or natural and inorganic or organic origin. They can be short or long, individual or organized, for example braided, or hollow or solid. They can have any shape and can in particular be circular or polygonal (square, hexagonal or octagonal) in cross section, according to the specific application envisaged. In particular, their ends are blunted and/or polished in order to avoid inflicting injury. The fibres have a length ranging from 1 μm to 10 mm preferably from 0.1 mm to 5 mm and better still from 0.3 mm to 3 mm. Their cross section can be included within a circle with a diameter ranging from 2 nm to 500 μm, preferably ranging from 100 nm to 100 μm and better still from 1 μm to 50 μm. Mention may be made, as fibres which can be used in the compositions according to the invention, of nonrigid fibres, such as polyamide (Nylon®) fibres, or rigid fibres, such as poly(imide-amide) fibres, for example those sold under the names Kermel® or Kermel Tech® by Rhodia, or poly(p-phenylene terephthalamide) (or aramid) fibres, in particular sold under the name Kevlar® by DuPont de Nemours,

and their mixtures.

According to one embodiment, the composition of the invention comprises at least one pulverulent colouring material, in particular at least one pigment.

Pulverulent Colouring Materials

A composition according to the invention can additionally comprise at least one pulverulent colouring material.

The pulverulent colouring materials can be present in a proportion of 0.01 to 30% by weight, in particular of 0.1 to 20% by weight and especially of 1 to 15% by weight, with respect to the total weight of the cosmetic composition.

A cosmetic composition in accordance with the invention can advantageously incorporate at least one colouring material chosen from organic or inorganic pulverulent colouring materials, in particular of pigment or pearlescent agent type, conventionally used in cosmetic compositions, materials with a specific optical effect, and their mixtures.

### Pigments

The term “pigments” should be understood as meaning white or coloured and inorganic or organic particles which are insoluble in aqueous solution and which are intended to colour and/or opacify the resulting film.

Mention may be made, as inorganic pigments which can be used in the invention, of titanium, zirconium or cerium oxides and also zinc, iron or chromium oxides, ferric blue, manganese violet, ultramarine blue and chromium hydrate.

Use is preferably made of pigments formed of iron oxides or titanium dioxide.

The pigment can also have a structure which can, for example, be of sericite/brown iron oxide/titanium dioxide/silica type. Such a pigment is sold, for example, under the reference Coverleaf NS or JS by Chemicals and Catalysts and exhibits a contrast ratio in the region of 30.

The colouring material can also comprise a pigment having a structure which can, for example, be of the type of silica microspheres comprising iron oxide. An example of a pigment exhibiting this structure is that sold by Miyoshi under the reference PC Ball PC-LL-100 P, this pigment being composed of silica microspheres comprising yellow iron oxide.

Mention may be made, among the organic pigments which can be used in the invention, of carbon black, pigments of D & C type, lakes based on cochineal carmine of barium, strontium, calcium or aluminum, or the diketopyrrolopyrroles (DPP) described in the documents EP-A-542 669, EP-A-787 730, EP-A-787 731 and WO-A-96/08537.

Advantageously, the pigments can be present in a hydrophobic coated form in the emulsion according to the invention. They are more particularly pigments treated at the surface with a hydrophobic agent in order to render them compatible with the fatty phase of the emulsion, in particular in order for them to have a good wettability with the oils of the fatty phase. Thus, these treated pigments are well dispersed in the fatty phase.

The hydrophobic treatment agent can be chosen from silicones, such as methicones, dimethicones or perfluoroalkylsilanes; fatty acids, such as stearic acid; metal soaps, such as aluminum dimyristate or the aluminum salt of hydrogenated tallow glutamate; perfluoroalkyl phosphates, perfluoroalkylsilanes, perfluoroalkylsilazanes, poly(hexafluoropropylene oxide)s, polyorganosiloxanes comprising perfluoroalkyl perfluoropolyether groups, amino acids; N-acylated amino acids or their salts; lecithin, isopropyl titanium triisostearate, and their mixtures.

The N-acylated amino acids can comprise an acyl group having from 8 to 22 carbon atoms, such as, for example, a 2-ethylhexanoyl, caproyl, lauroyl, myristoyl, palmitoyl, stearoyl or cocoyl group. The salts of these compounds can be aluminum, magnesium, calcium, zirconium, zinc, sodium or potassium salts. The amino acid can, for example, be lysine, glutamic acid or alanine.

The term “alkyl” mentioned in the abovementioned compounds denotes in particular an alkyl group having from 1 to 30 carbon atoms and preferably having from 5 to 16 carbon atoms.

Hydrophobic treated pigments are described in particular in Application EP-A-1 086 683.

The pigments can be present in a content ranging from 0.01 to 20% by weight, with respect to the total weight of the composition, preferably in a content at least equal to 5% by weight, in particular ranging from 5 to 20% by weight, more particularly from 8 to 20% by weight, and preferentially ranging from 8 to 15% by weight.

### Pearlescent Agents

The term “pearlescent agents” should be understood as meaning coloured particles of any shape, which may or may not be iridescent, produced in particular by certain shellfish in their shells or synthesized, which exhibit a colouring effect by optical interference.

The pearlescent agents can be chosen from pearlescent pigments, such as titanium oxide-coated mica covered with an iron oxide, titanium oxide-coated mica covered with bismuth oxychloride, titanium oxide-coated mica covered with chromium oxide or titanium oxide-coated mica covered with an organic dye, and pearlescent pigments based on bismuth oxychloride. They can also be mica particles, at the surface of which at least two successive layers of metal oxides and/or of organic colouring materials are superimposed.

Mention may also be made as examples of pearlescent agents, of natural mica covered with titanium oxide, iron oxide, natural pigment or bismuth oxychloride.

Materials with an Optical Effect

The cosmetic composition according to the invention can also comprise at least one material with a specific optical effect.

This effect is different from a simple conventional colouring effect, that is to say a unified and stabilized effect, such as that produced by conventional colouring materials, such as, for example, monochromatic pigments. Within the meaning of the invention, the term “stabilized” means devoid of an effect of variability in the colour with the angle of observation or else in response to a change in temperature.

For example, this material can be chosen from particles with a metallic glint, goniochromatic colouring agents, diffracting pigments, thermochromic agents, optical brighteners and fibres, in particular interference fibres. Of course, these various materials can be combined so as to provide the simultaneous display of two effects, indeed even of a novel effect in accordance with the invention.

The particles with a metallic glint which can be used in the invention are chosen in particular from:

particles of at least one metal and/or of at least one metal derivative,

particles comprising an organic or inorganic substrate, made of one or more materials, at least partially covered with at least one layer with a metallic glint comprising at least one metal and/or at least one metal derivative, and

mixtures of the said particles.

Mention may be made, among the metals which can be present in the said particles, for example, of Ag, Au, Cu, Al, Ni, Sn, Mg, Cr, Mo, Ti, Zr, Pt, Va, Rb, W, Zn, Ge, Te, Se and their mixtures or alloys. Ag, Au, Cu, Al, Zn, Ni, Mo, Cr, and their mixtures or alloys (for example, bronzes and brasses) are preferred metals.

The term “metal derivatives” denotes compounds derived from metals, in particular oxides, fluorides, chlorides and sulphides.

The goniochromatic colouring agent can be chosen, for example, from interference multilayer structures and liquid crystal colouring agents.

### Cosmetic Adjuvants

The compositions of the invention can additionally comprise one or more of the adjuvants known in the cosmetic and dermatological fields, such as moisturizing agents; emollients; hydrophilic or lipophilic active principles; agents for combating free radicals; sequestering agents; antioxidants; preservatives; basifying or acidifying agents; fragrances; hydrophilic gelling agents; film-forming agents, in particular film-forming polymers (for compositions in a sustained direction), organic or physical sunscreens, water-soluble or fat-soluble dyes; and their mixtures. The amount of these various adjuvants are those conventionally used in foundations.

Of course, the person skilled in the art will take care to choose the optional adjuvant or adjuvants added to the composition according to the invention so that the advantageous properties intrinsically attached to the composition in accordance with the invention are not, or not substantially, detrimentally affected by the envisaged addition.

These adjuvants are generally present in the composition in a content ranging from 0.01 to 20% by weight, preferably from 0.1 to 10% by weight, with respect to the total weight of the said composition.

The invention is illustrated in the examples presented below by way of illustration and without implied limitation of the scope of the invention:

Unless otherwise indicated, the values in the examples below are expressed in % by weight, with respect to the total weight of the composition.

### EXAMPLES

### Examples 1 and 2

### Foundation: Influence of the viscosity (W/O Comprising a Silicone Oily Phase)

| | | Example 1 | Example 2 |
|---|---|---|---|
| | | (invention) | (comparative) |
| | | % by weight | % by weight |
| A | Dimethicone copolyol, sold | 1.4881 | 1.9981 |
| | under the reference KF 6017 | | |
| | by Shin Etsu | | |
| | Bis PEG/PPG-14/14 | 0.66 | 1.00 |
| | dimethicone + | | |
| | cyclopentasiloxane, sold | | |
| | under the reference Abil EM | | |
| | 97 by Goldschmidt | | |
| | Cyclopentasiloxane | 10.9084 | 17.6484 |
| | Phenyl trimethicone, sold | 1.31 | 2.00 |
| | under the reference DC556 | | |
| | by Dow Corning | | |
| | Ethylhexyl methoxycinnamate | 1.97 | 3.00 |
| | Squalane | 0.66 | 1.00 |
| B | Cyclopentasiloxane | 7.00 | 7.00 |
| | Yellow iron oxide coated | 1.1715 | 1.1715 |
| | with aluminium stearoyl | | |
| | glutamate (3%) | | |
| | NAI-C33-9001-10 from | | |
| | Miyoshi Kasei | | |
| | Red iron oxide coated with | 0.3430 | 0.3430 |
| | aluminium stearoyl | | |
| | glutamate (3%) | | |
| | NAI-C33-8001-10 from | | |
| | Miyoshi Kasei | | |
| | Black iron oxide coated | 0.108 | 0.108 |
| | with aluminium stearoyl | | |
| | glutamate (3%) | | |
| | NAI-C33-7001-10 from | | |
| | Miyoshi Kasei | | |
| | Titanium dioxide (anatase) | 10.381 | 10.381 |
| | coated with aluminium | | |
| | stearoyl glutamate (97/3) | | |
| | NAI-TAO-77891 from Miyoshi | | |
| | Kasei | | |
| C | Talc, sold under the | 0.33 | 0.50 |
| | reference Micro Ace P3 by | | |
| | Nippon Talc | | |
| | Nylon 12 powder, sold under | 0.33 | 0.50 |
| | the reference SP 500 by | | |
| | Toray | | |
| D | Demineralized water | 46.14 | 36.15 |
| | Butylene glycol | 3.00 | 3.00 |
| | Magnesium sulphate | 0.70 | 0.70 |
| | Maltitol + sorbitol, sold | 0.50 | 0.50 |
| | under the reference Mabit | | |
| | by Hayashibara | | |
| E | Ethanol | 13.00 | 13.00 |
| | TOTAL | 100% | 100% |

### Procedure

The constituents of the phase A are weighed out into the main beaker and stirring is carried out under a Moritz stirrer (1000 rev/min) while maintaining at ambient temperature.

The phase B is prepared simultaneously by milling the mixture of pigments and of cyclopentasiloxane three times on a triple roll mill.

This phase B is subsequently added while continuing to stir, along with the fillers (phase C).

The aqueous phase D is also prepared separately by weighing the butylene glycol, the magnesium sulphate, the maltitol+sorbitol and the water into a beaker at ambient temperature.

The aqueous phase D is stirred using a magnetic bar until homogeneous.

The emulsion is prepared at ambient temperature: the aqueous phase D is run into the fatty phase while gradually increasing the stirring speed (Moritz) up to 4000 revolutions/min. Stirring is maintained for 7 min.

Finally, the phase E (ethanol) is then added during the final 3 minutes of the emulsification.

### Viscosity Results

| | Example 1 | Example 2 |
|---|---|---|
| Viscosity (Pa · s) | (Invention) | (Comparative) |
| Low gradient | 105 | 66.4 |
| 0.01 s⁻¹ | | |
| High gradient | 0.28 | 0.18 |
| 1000 s⁻¹ | | |

### Sensory Evaluation

In the case of Example 1 (invention), the applicator as described in FIGS. 1 and 2 (orifice of 3 mm) starts perfectly: the pressure to be exerted on the reservoir of the applicator is low and the amount of foundation delivered to the roller is controlled. The amount of foundation released by the roller when it is applied to the skin is appropriate and the makeup result is homogeneous.

On the other hand, in the case of Example 2 (comparative), despite the applicator being easy to start, the amount of foundation released onto the roller is too great, resulting in the foundation running off the head of the applicator. This results in a makeup result which is less homogeneous than in the case of Example 1.

### Example 3

### Foundation (W/O Comprising a Hydrocarbon-Comprising Oily Phase)

| | | % by |
|---|---|---|
| | | weight |
| A1 | PEG-30 dipolyhydroxystearate, sold under | 2.98 |
| | the reference Arlacel P 135 by Uniqema | |
| | Tristearin + acetylated ethylene glycol | 0.52 |
| | stearate, sold under the reference Unitwix | |
| | by United Guardian | |
| | Dicapryl carbonate | 2.24 |
| | Dipentaerythrityl hexacaprylate/caprate, | 7.46 |
| | sold under the reference DUB DPHCC by | |
| | Stearinerie Dubois | |
| | Ethylhexyl methoxycinnamate | 5.22 |
| | Isodecyl neopentanoate | 4.55 |
| | Bentone gel, sold under the referene | 3.84 |
| | Bentone Gel IHD V by Elementis | |
| A2 | Isohexadecane | 5.63 |
| | Yellow iron oxide coated with aluminium | 1.6 |
| | stearoyl glutamate (3%) | |
| | NAI-C33-9001-10 from Miyoshi Kasei | |
| | Red iron oxide coated with aluminium | 0.45 |
| | stearoyl glutamate (3%) | |
| | NAI-C33-8001-10 from Miyoshi Kasei | |
| | Black iron oxide coated with aluminium | 0.11 |
| | stearoyl glutamate (3%) | |
| | NAI-C33-7001-10 from Miyoshi Kasei | |
| | Titanium dioxide (anatase) coated with | 9.84 |
| | aluminium stearoyl glutamate (97/3) | |
| | NAI-TAO-77891 from Miyoshi Kasei | |
| A3 | Methyl methacrylate/ethylene glycol | 3.00 |
| | dimethacrylate copolymer, sold under the | |
| | reference Techpolymer MBP-8 by Sekisui | |
| | Plastics | |
| A4 | Tocopherol acetate | 0.075 |
| B | Demineralized water | 46.485 |
| | Glycerol | 5.00 |
| | Methylparaben | 0.3 |
| | Magnesium sulphate | 0.60 |
| | EDTA | 0.10 |
| | TOTAL | 100% |

### Procedure

The constituents of the phase A1, with the exception of the bentone gel, are weighed out into the main beaker and the beaker is placed on a water bath until the wax has melted.

The bentone gel is subsequently added at ambient temperature and stirring is carried out using a Moritz stirrer (1000 rev/min).

The phase A2 is prepared separately by milling the mixture of pigments and of isohexadecane three times on a triple roll mill.

This phase A2 is subsequently added at ambient temperature while continuing to stir, along with the phases A3 and A4.

The aqueous phase B is also prepared separately by weighing, into a beaker, the glycerol, the magnesium sulphate, the EDTA and the methylparaben and by adding the water preheated to 95° C.

The aqueous phase B is stirred using a magnetic bar until homogeneous.

The emulsion is prepared at ambient temperature: the aqueous phase B is run into the fatty phase while gradually increasing the stirring speed (Moritz) up to 4000 revolutions/min. Stirring is maintained for 10 min.

### Viscosity Results

| Viscosity (Pa · s) | Example 3 (Invention) |
|---|---|
| Low gradient | 368 |
| 0.01 s⁻¹ | |
| High gradient | 0.45 |
| 1000 s⁻¹ | |

### Sensory Evaluation

As in the case of Example 1, the applicator as described in FIGS. 1 and 2 (orifice 3 mm) starts very satisfactorily: the pressure to be exerted on the reservoir of the applicator is low and the amount of foundation delivered onto the roller is controlled. Furthermore, the amount of foundation released by the roller when it is applied to the skin is appropriate and the makeup result is homogeneous.

Examples 1 (W/O comprising a silicone oily phase) and 3 (W/O comprising a hydrocarbon-comprising oily phase) together show that the nature of the oils in the oily phase, silicone or hydrocarbon-comprising, has no or little impact on the result: it is the rheological profile of the composition which is important.

The above written description of the invention provides a manner and process of making and using it such that any person skilled in this art is enabled to make and use the same, this enablement being provided in particular for the subject matter of the appended claims, which make up a part of the original description.

As used herein, the words “a” and “an” and the like carry the meaning of “one or more.”

The phrases “selected from the group consisting of,” “chosen from,” and the like include mixtures of the specified materials. Terms such as “contain(s)” and the like are open terms meaning ‘including at least’ unless otherwise specifically noted.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A cosmetic packaging and application combination for making up and/or caring for a keratinous substance comprising:
a container comprising at least one cosmetic composition in the water-in-oil emulsion form, having the following rheological profile:
a viscosity of between 35 and 1000 Pa·s at a shear gradient of 0.01 s⁻¹,
a viscosity of between 0.20 and 0.90 Pa·s at a shear gradient of 1000 s⁻¹,
as measured using a controlled-stress rheometer, AR-G2, TA Instruments, at 25° C., and
an applicator head integral with the container and supporting a roller, the roller comprising an application surface capable, in response to its movement on engaging with the keratinous substance, of rotating about at least one axis or centre of rotation, the application surface being in liquid communication with the container via at least one passage which, over at least a portion of its length, exhibits a diameter of 1 to 6 mm.

2. The cosmetic packaging and application combination according to claim 1, in which the passage emerges with regard to the application surface via at least one orifice exhibiting a diameter of 1 to 6 mm.

3. The cosmetic packaging and application combination according to claim 1, in which the composition comprises from 10 to 80% by weight of aqueous phase, with respect to the total weight of the said composition.

4. The cosmetic packaging and application combination according to claim 1, in which the composition comprises from 10 to 90% by weight of liquid fatty phase, with respect to the total weight of the composition.

5. The cosmetic packaging and application combination according to claim 4, in which the liquid fatty phase comprises oils chosen from volatile or nonvolatile oils of hydrocarbon-comprising or silicone type, and their mixtures.

6. The cosmetic packaging and application combination according to claim 1, in which the composition comprises at least one surfactant chosen from amphoteric, anionic, cationic and nonionic surfactants, used alone or as mixtures.

7. The cosmetic packaging and application combination according to claim 6, in which the surfactant(s) is/are present in a total content ranging from 0.5 to 15% by weight, with respect to the total weight of the composition.

8. The cosmetic packaging and application combination according to claim 1, in which the composition comprises at least one agent chosen from inorganic or organic lipophilic gelling agents, lipophilic structuring agents, particulate materials chosen from fillers, pulverulent colouring materials and their mixtures.

9. The cosmetic packaging and application combination according to claim 1, in which the composition comprises at least one particulate material chosen from pigments, fillers and their mixtures.

10. The cosmetic packaging and application combination according to claim 1, the composition constituting a blusher, an eyeshadow, a foundation, a concealer, a lipstick, a product for making up the body, a product for caring for the face, a product for caring for the body or an antisun product.

11. The cosmetic packaging and application combination according to claim 1, the composition constituting a foundation.

12. The cosmetic packaging and application combination according to claim 1, in which the roller has an application surface of the following type: foam comprising open or closed cells which are optionally flocked, flocking, elastomer, sintered material, woven material or nonwoven material.

13. A method for making up and/or caring for a keratinous substance, comprising contacting the keratinous substance with the cosmetic packaging and application combination according to claim 1 via said roller and applying said composition to said keratinous substance through said roller.

14. The method according to claim 13, wherein said keratinous substance is the skin of the face.

15. The method according to claim 13, wherein said composition is a foundation
